SEQUENCE LISTING (1 sequence)

SEQ ID NO: 1 - protein, 9 aa, Homo sapiens
CKGGRAKDC

(19) United States
(12) Patent Application Publication
(10) Pub. No.: US 2006/0122118 A1
(43) Pub. Date: Jun. 8, 2006

(54) Therapeutic compositions and methods of using same

(76) Inventor: Siew P. Ho, Chadds Ford, PA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/293,581

(22) Filed: Dec. 2, 2005

Related U.S. Application Data
(60) Provisional application No. 60/632,864, filed on Dec. 3, 2004.

(51) Int. Cl. A61K 48/00; A61K 38/17; A61K 38/18

(52) U.S. Cl. 514/12; 514/44

(57) ABSTRACT

A targeted composition comprising a modulation molecule, such as an siRNA oligo or a small molecule, and a translocation peptide is disclosed. Methods of making and using the composition are also disclosed. The disclosed composition can be employed in a variety of therapeutic and research applications, in which it is desirable to deliver a modulation molecule, such as an siRNA oligo or a small molecule, to a particular site, such as a site of vascularization. In one aspect of a composition of the invention, a translocation peptide directs the modulation molecule to a selected location, and a modulation molecule modulates the expression or activity of a selected target disposed at or near the selected location.

### CROSS-REFERENCE TO RELATED APPLICATIONS

This patent application claims benefit to provisional application U.S. Ser. No. 60/632,864 filed Dec. 3, 2004. The entire teaching of the referenced application is incorporated herein by reference.

### FIELD OF THE INVENTION

The present invention relates generally to compositions and methods for treating a condition by modulating the activity of a selected target, or an activity mediated by a selected target. In various aspects, the invention also relates to siRNA-mediated therapeutic treatment regimes and compositions for use in such regimes. The present invention also relates to target validation methods. The compositions of the present invention comprise a modulation molecule and a translocation peptide.

### BACKGROUND OF THE INVENTION

Since its discovery, there has been a great deal of interest in the field of RNA interference (“RNAi”). The field has been reported to hold great therapeutic potential. RNA interference generally refers to the process of sequence-specific post-transcriptional gene silencing mediated by double-stranded RNAs (dsRNAs), which was apparently first reported by Fire et al. (Fire et al., (1998) Nature 391:806).

Currently, the process of RNAi-mediated gene silencing is believed to follow a general mechanism: the presence of long dsRNAs in cells stimulates the activity of an enzyme known as “Dicer.” Dicer clips the dsRNA into short pieces of dsRNA known as short interfering RNAs (siRNAs). These siRNAs are typically about 21 to about 23 nucleotides in length. One or more of the siRNAs generated by Dicer is then incorporated into a complex having endonuclease activity, known as the RNA-induced silencing complex (“RISC”), which mediates cleavage of single-stranded RNA having sequence complementary to one of the strands of the siRNA duplex.

A current challenge associated with the use of RNAi methodology in therapeutic applications is the rapid clearance of the RNAi reagent (i.e., the dsRNA oligo) from the circulation of a subject. Using present methodology, most of the RNAi reagent introduced using conventional routes of administration such as intravenous delivery is cleared from the circulation by first pass metabolism. Consequently, little to no suppression of the intended RNA target is observed in vivo. To date, this and other hurdles has led to the effective application of RNAi technology in only limited circumstances, such as the treatment of macular degeneration, in which the reagent is not introduced into the circulation of the subject but is injected directly into the eye of a subject.

Thus, what is needed is a method of treating a condition that takes advantage of a targeting moiety, such as a peptide, to direct the RNAi oligo to a site at which gene silencing can be effectively achieved.

### SUMMARY OF THE INVENTION

In one aspect of the present invention a composition comprising a translocation peptide and a modulation molecule directed to a selected target is disclosed. In various embodiments of the composition, the selected target can be an angiogenic protein, and can be selected from the group consisting of VEGF and/or a VEGFR. The translocation peptide can be vasculature translocation peptide and can comprise CKGGRAKDC (SEQ ID NO:1). The modulation molecule can be selected from the group consisting of a double-stranded siRNA oligo, a PNA, a single-stranded antisense oligo, a peptide, an aptamer and a pyrrole-imidazole oligomer. The selected target can be any target, such as VEGF or a VEGF receptor.

A composition of the present invention can further comprise a linker. The linker can be any kind of linker; for example the linker can be selected from the group consisting of a chemical linkage, a peptide linker, a nucleic acid linker and a PNA linker. When the linker is a chemical linkage, it can be selected from the group consisting of a disulfide linkage, a urethane linkage, an ester linkage, and an amide linkage, for example.

A composition of the present invention can further comprise a detectable reagent and can be selected, for example, from the group consisting of a fluorophore and a radiolabel. When the detectable reagent is a fluorophore, the fluorophore can be selected from the group consisting of fluorescein, Cy-3 and an ALEXA dye.

In another aspect of the present invention, a method of down-regulating the expression of a selected target comprising administering to a subject the composition of claim 1 is disclosed. In various embodiments of the method, the selected target can be an angiogenic protein, and can be selected from the group consisting of VEGF and a VEGFR. The translocation peptide can be a translocation peptide directed to a vascular structure and can comprise SEQ ID NO:1. The modulation molecule can be selected from the group consisting of a double-stranded siRNA oligo, a PNA, a single-stranded antisense oligo and a peptide. The selected target can be any target, such as VEGF or the VEGF receptor.

The composition employed in the method can further comprise a linker. The linker can be any kind of linker; for example the linker can be selected from the group consisting of a chemical linkage, a peptide linker, a nucleic acid linker and a PNA linker. When the linker is a chemical linkage, it can be selected from the group consisting of a disulfide linkage, a urethane linkage, an ester linkage, and an amide linkage.

The composition employed in the method can further comprise a detectable reagent and can be selected, for example, from the group consisting of a fluorophore and a radiolabel. When the detectable reagent is a fluorophore, the fluorophore can be selected from the group consisting of TAMRA, BODIPY, cyanin derivatives such as Cy3 or Cy5, fluorescein isothiocyanate, Texas red, rhodamine, dansyl, umbelliferone and an ALEXA dye.

In another aspect of the present invention, a method of treating a condition comprising administering to a subject in need thereof the composition of claim 1 is disclosed. In various embodiments of the method, the selected target can be an angiogenic protein, and can be selected from the group consisting of VEGF and a VEGFR. The translocation peptide can be an angiogenic protein translocation peptide and can comprise CKGGRAKDC (SEQ ID NO: 1). The modulation molecule can be selected from the group consisting of a double-stranded siRNA oligo, a PNA, a single-stranded antisense oligo and a peptide. When the modulation molecule is a double-stranded siRNA oligo, the double-stranded siRNA oligo comprises SEQ ID NO:2. The selected target can be any target, such as VEGF or the VEGF receptor.

The composition employed in the method can further comprise a detectable reagent and can be selected, for example, from the group consisting of a fluorophore and a radiolabel. When the detectable reagent is a fluorophore, the fluorophore can be selected from the group consisting of TAMRA, BODIPY, cyanin derivatives such as Cy3 or Cy5, fluorescein isothiocyanate, Texas red, rhodamine, dansyl, umbelliferone and an ALEXA dye.

### BRIEF DESCRIPTION OF THE DRAWINGS

FIG. 1 is a schematic depicting several configurations of a composition of the present invention. In the figure, the cross-hatched rectangle represents a translocation peptide and the open rectangle represents a modulation molecule. Two exemplary configurations of the composition are depicted, although others are possible.

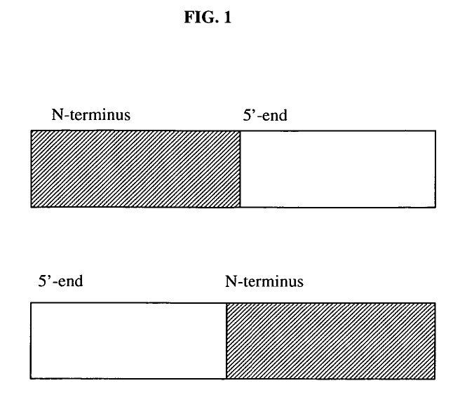

FIG. 2 is a schematic depicting several configurations of a composition of the present invention. In the figure, the cross-hatched rectangle represents a translocation peptide, the open rectangle represents a modulation molecule and the vertically-hatched rectangle represents a linker. Two exemplary configurations of the composition are depicted, although others are possible.

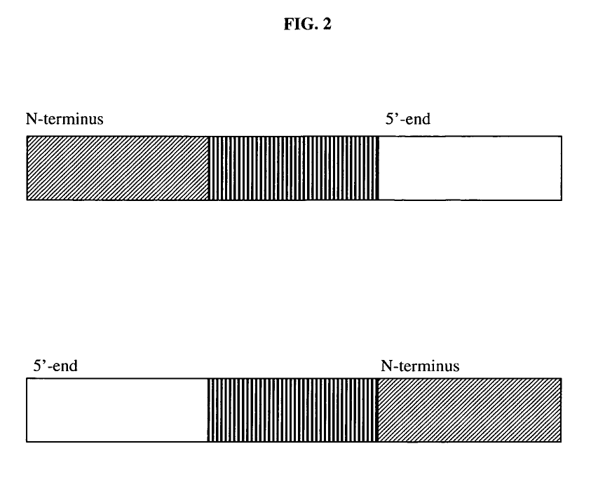

FIG. 3 is a schematic depicting several configurations of a composition of the present invention. In the figure, the cross-hatched rectangle represents a translocation peptide, the open rectangle represents a modulation molecule, the vertically-hatched rectangle represents a linker and the stippled rectangle represents a detectable label. Four exemplary configurations of the composition are depicted, although others are possible.

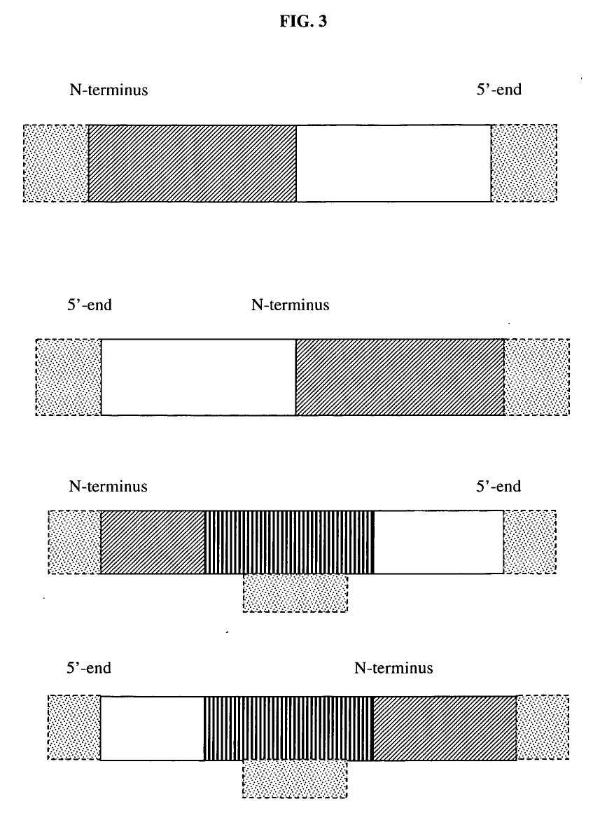

### DETAILED DESCRIPTION OF THE INVENTION

The present invention generally relates to methods and compositions for modulating the expression of a selected target and for methods and compositions for the treatment of a condition associated with the expression of the selected target. In one embodiment, a modulation molecule, such as an siRNA oligo, is joined with a translocation peptide. The translocation peptide can steer the composition to any tissue; for example, peptides targeting vascular tissue, are disclosed in one embodiment of the present invention. The modulation molecule and the translocation peptide can optionally be joined by a linker, which can be any chemical compound, such as a peptide or a small molecule. A composition of the present invention can also comprise a detectable label, such as a fluorophore. The incorporation of a detectable label can facilitate the tracking of the composition, for example by fluorescence microscopy.

The compositions of the present invention can be employed in a range of different applications, such as modulating the expression of a selected target, such as a protein or mRNA, or the treatment of a condition, such as a condition associated with the expression, overexpression or underexpression of a selected target or the modulation of an activity of, or an activity that is mediated by, a selected target. The precise components of the composition can be a factor in determining the application for which the composition can be employed.

### I. Definitions

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or less (e.g., ±15%, ±10%, ±7%, ±5%, ±4%, ±3%, ±2%, ±1%, or ±0.1%) from the specified amount, as such variations are appropriate.

As used herein, the term “RNA” or “RNA molecule” or “ribonucleic acid molecule” means a polymer of ribonucleotides. The term “DNA” or “DNA molecule” or “deoxyribonucleic acid molecule” refers to a polymer of deoxyribonucleotides. In the context of the present invention, DNA and RNA can be isolated from recombinant or non-recombinant sources, or DNA and RNA can also be chemically synthesized. DNA and RNA can be single-stranded (i.e., ssRNA and ssDNA, respectively) or multi-stranded (e.g., double stranded, i.e., dsRNA and dsDNA, respectively or triple stranded). In the context of the present invention, nucleotides making up any nucleic acid sequence, including DNA or RNA sequences optionally comprising modified nucleotides, can be of any length and can be comprised entirely of naturally occurring nucleotides (i.e., the standard nucleotides, G, A, T, C and U), entirely of modified nucleotides or nucleotide analogs, or a combination of naturally occurring nucleotides and modified nucleotides or nucleotide analogs in any proportion.

As used herein, the term “small interfering RNA” (“siRNA”) (also referred to in the art as “short interfering RNAs”) means an RNA oligomer comprising between about 10 and about 50 nucleotides, and preferably about 21-23 nucleotides, which is capable of directing or mediating RNA interference.

As used herein, the term “nucleotide analog”, also referred to herein as a “modified nucleotide,” means a non-standard nucleotide, including non-naturally occurring ribonucleotides or deoxyribonucleotides. A nucleotide analog can be disposed in an RNA sequence or a DNA sequence. Examples of nucleotide analogs include a methylenediol, ethylene diol, oxymethylthio, oxyethylthio, oxycarbonyloxy, phosphorodiamidate, phophoroamidate, and/or phosphorothioate linkages. In the case of an RNA analog, for RNAi applications, it is generally preferable that the analog retains the ability to mediate RNA interference. “Modified nuclcleotides” include modified bases including, for example, tritylated bases, propynyl bases, 4-thiouracil and unusual bases, such as inosine, and modified sugars, such as 2′-methoxy and 2′-fluoro, locked nucleic acids, 2′-methoxyethyl, 2′-amino and morpholino compounds.

As used herein, the terms “nucleotide”, “base” and “nucleic acid” are used interchangeably and are equivalent. Additionally, the terms “nucleotide sequence”, “nucleic acid sequence”, “nucleic acid molecule” and “segment” are used interchangeably and are equivalent. The terms “nucleotide”, “base”, “nucleic acid”, “nucleotide sequence”, “nucleic acid sequence”, “nucleic acid molecule” and “segment” encompass any deoxyribonucleic acid (DNA), ribonucleic acid (RNA), oligonucleotides, sequences generated by the polymerase chain reaction (PCR), and fragments generated by ligation, scission, endonuclease action, exonuclease action or other mechanism. Additionally, the terms encompass single mers that are not part of a sequence or fragment.

As used herein, the terms “oligonucleotide” and “polynucleotide” are used interchangeably with the term “oligo” and means a short, contiguous sequence comprising about 50 or fewer nucleotides, nucleotide analogs or a combination thereof. An oligonucleotide or a polynucleotide can be naturally occurring or synthetic.

An oligo of the present invention can comprise any polyribonucleotide or polydeoxribonucleotide, and can comprise unmodified RNA or DNA or modified RNA or DNA. For example, an oligo can comprise single- and double-stranded DNA, DNA that is a mixture of single- and double-stranded regions, single- and double-stranded RNA, RNA that is mixture of single- and double-stranded regions, hybrid molecules comprising DNA and RNA that can be single-stranded or, more typically, double-stranded or a mixture of single- and double-stranded regions. In addition, an oligo can comprise triple-stranded regions comprising RNA or DNA or both RNA and DNA. An oligo can also contain one or more modified bases or DNA or RNA backbones modified for stability or for other reasons.

As used herein, the term “selected target” means a DNA sequence encoding, an mRNA sequence encoding, or a protein encoded by, a gene for which it is desired to selectively modulate (e.g., up-regulate, down-regulate or silence) expression or activity. The term “activity” encompasses any activity of the selected target, or any activity mediated by the selected target.

As used herein, the term “modulation molecule” means any agent, such as a peptide, one or more nucleotides, a single or double-stranded nucleic acid sequence, a PNA, a pyrrole-imidazome oligomer, a small molecule, an antisense oligo, an aptamer, a polypyrrole oligomer or a polyimidazole oligomer, that is known or suspected to promote the modulation of the expression, activity, or activity mediated by, a selected target in vivo or in vitro. A modulation molecule can be disposed in a composition. When a modulation molecule is a peptide, it can comprise an amino acid sequence identical or similar to an amino acid sequence that is found in vivo. Alternatively, when a modulation molecule is a peptide it can comprise an amino acid sequence that is not found normally found in vivo.

A modulation molecule can be synthesized or isolated from a biological sample, either by itself or as a product of the cleavage of a larger protein or nucleic acid sequence. A modulation molecule can comprise naturally-occurring amino acids or nucleotides, or a modulation molecule can also comprise non-standard and/or non-naturally occurring amino acids or nucleotides.

As used herein, the terms “protein”, “polypeptide” and “peptide” are used interchangeably and mean any polymer comprising any of the 20 naturally occurring biological amino acids, regardless of its size. Although “protein” is typically used to refer to relatively large polypeptides, and “peptide” is typically used to refer to small polypeptides, the usage of these terms in the art overlaps and varies. Therefore, the term “polypeptide” as used herein refers to peptides, polypeptides and proteins, unless otherwise noted. The terms “protein”, “polypeptide” and “peptide” are used interchangeably herein.

As used herein, the terms “amino acid,” “amino acid residue” and “residue” are used interchangeably and mean any of the twenty naturally occurring amino acids. The amino acid residues described herein are preferably in the “L” isomeric form. However, residues in the “D” isomeric form can be substituted for any L-amino acid residue, as long as the desired functional property is retained by the polypeptide (e.g., enzymatic activity). NH₂ refers to the free amino group present at the amino terminus of a polypeptide. COOH refers to the free carboxy group present at the carboxy terminus of a polypeptide. In addition, the phrases “amino acid” and “amino acid residue” are broadly defined herein to include modified and unusual amino acids.

As used herein the term “complementary” means a nucleic acid sequence that is base paired, or is capable of base-pairing, according to the standard Watson-Crick complementarity rules. These rules generally hold that guanine pairs with cytosine (G:C) and adenine pairs with either thymine (A:T) in the case of DNA, or adenine pairs with uracil (A:U) in the case of RNA. The term complementary also includes nucleic acid sequences in which an inosine is present, in which case the inosine can pair with C, A or U.

As used herein, the terms “isolated” and “purified” are used interchangeably and refer to material (e.g., a nucleic acid or a polypeptide) removed from its original environment (e.g., the natural environment, if it is naturally occurring), and thus is altered “by the hand of man” from its natural state. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide and/or protein sequences of the present invention; such sequences are excluded from the scope of the present invention.

As used herein, the term “linker” means any chemical moiety that joins a translocation peptide and an modulation molecule. A linker can comprise any chemical moiety, for example, a small molecule, a nucleic acid, a peptide, or simply a sequence of one or more atoms or functional groups joined by chemical bonds.

As used herein, the term “translocation peptide” means any peptide that has the ability to steer a composition to a desired location. There is no limit on the mechanism by which a translocation peptide steers a composition to a desired location. For example, a translocation peptide can facilitate insertion and/or translocation across the membrane of a cell In another example, a translocation peptide can facilitate the association of the composition, or a component thereof, with a selected target. A translocation peptide can be of any length or amino acid composition, and can include non-standard amino acids or amino acid analogs.

### II. Compositions of the Present Invention

In one embodiment of the present invention, a composition of the present invention comprises a modulation molecule and a translocation peptide. As described herein, these components can be joined in the composition in any order. The various possible components of a composition of the present invention are discussed in further detail below.

### II.A. Modulation Molecule

One component of a composition of the present invention is a modulation molecule directed against a selected target. A selected target can be any protein for which it is desired to modulate (e.g., silence, up- or down-regulate) activity or expression. Typically, but not necessarily, a selected target will be a nucleic acid sequence encoding a protein involved in a disease condition. For example, a selected target can comprise a nucleic acid sequence, e.g., a gene, encoding a protein known or suspected to be involved in an angiogenic process. A non-limiting list of preferred selected targets include a nucleic sequence encoding VEGF or encoding a VEGF receptor (VEGFR). Other examples of selected targets that can be employed in the present invention include nucleic acids encoding proteins implicated in angiogenesis, such as basic fibroblast growth factor. Other representative selected targets include angiopoeitins and integrins.

A modulation molecule can be any agent that preferentially associates with a selected target or a nucleic acid encoding a selected target, to the exclusion of any other nucleic acid, protein or other chemical species present in a milieu in which the protein or nucleic acid is disposed. A modulation molecule can also include molecules such as aptamers. An aptamer is a molecule that achieves a therapeutic effect by binding to a protein or other biomolecule. For example an aptamer can achieve a therapeutic effect by binding to a protein, such as a transcription factor, which in turn inhibits transcription of a nucleic acid by blocking the association of the transcription factor and the nucleic acid. Thus, a modulation molecule of the present invention can achieve a desired result, e.g., modulation of the expression or activity of a selected target, regardless of the mechanism by which the result is achieved.

Preferably, a modulation molecule directed against a selected target is known to have the ability to modulate (e.g., up- or down-regulate or silence) the activity or expression of a nucleic acid encoding a selected target. Alternatively, a composition of the present invention can be employed in an exploratory experiment and it may not be known whether the modulation molecule has the ability to down-regulate or silence a selected target. Expression of a selected target can be determined using standard assays, such as real-time PCR or western blotting; other appropriate expression assays will be known to those of ordinary skill in the art. When a selected target is a protein, activity assays will be dependent on the identity and properties of the selected target.

A modulation molecule directed against a selected target can comprise any of a range of chemical species. In one embodiment, a modulation molecule directed against a selected target is an siRNA oligo known or suspected to modulate expression of a selected target..

An siRNA oligo of the present invention can be of any length, although oligos of about 20 to 25 nucleotides in length are generally preferable. The siRNA can be single stranded or double stranded and can be chemically synthesized, for example using an automated nucleic acid synthesizer, or the siRNA can be isolated intact from a biological source. Alternatively, the siRNA can be prepared from a larger nucleic acid sequence by cleaving the larger sequence into smaller sequences using a restriction enzyme or other means.

An siRNA of the present invention preferably has known RNAi activity against a particular protein. There is no limit on the identity of a protein against which an siRNA is directed; validated therapeutic targets are generally preferred, although the compositions of the present invention can also be employed in target validation protocols. An siRNA of the present invention comprises a strand that is complementary to a region of a sequence encoding a protein for which it is desired to silence, increase or decrease expression. The expression or expression level of the protein can be implicated in a disease condition.

By way of example, in one embodiment of the present invention, the siRNA is directed to a nucleotide sequence encoding VEGF or a VEGF receptor. VEGF is a protein implicated in angiogenesis, can be a therapeutic target implicated in a variety of conditions, such as cancer and obesity. VEGF is produced by normal cell lines and tumor cell lines, and induces plasminogen activators in endothelial cells, which are subsequently involved in the proteolytic degradation of extracellular matrix during the formation of capillaries. VEGF represents a family of proteins, including various forms of VEGF, and a nucleotide encoding any member of the a VEGF family can serve as an siRNA target in the present invention.

An anti-VEGF or anti-VEGFR siRNA can therefore be used to silence or decrease expression of VEGF or VEGFR, thereby slowing or halting angiogenesis. Although a nucleotide sequence encoding any protein can be targeted by an siRNA of the present invention, a non-limiting list of examples in addition to VEGF and VEGFR includes basic fibroblast growth factor, angiopoeitins and integrins.

It is noted that an siRNA of the present invention can be double stranded. When an siRNA is double stranded, it can comprise two strands of RNA or it can comprise strands of modified RNA or DNA such that they retain the ability to induce RNA interference.

When designing an siRNA oligo of the present invention, a gene is first selected for silencing. At least part of the selected gene's sequence should be available. Examples of suitable genes are provided herein. When the siRNA oligo is double stranded, the first strand should be complementary to a portion of this gene sequence, and the other strand is identical or substantially identical to the same portion of the gene.

In another embodiment of the present invention, a modulation molecule is a polyamide nucleic acid, also equivalently referred to herein as a peptide nucleic acid (PNA). PNAs are modified nucleic acids in which the sugar phosphate skeleton of a nucleic acid has been converted to an aminoethylglycine (e.g., N-(2-aminoethyl)glycine) skeleton. The nucleic acids are retained and are bound directly or indirectly to aza nitrogen atoms of the amide portion of the backbone. Due to this unique chemistry, PNAs do not exhibit electrostatic repulsion and therefore have an increased ability to form double strands as compared with conventional nucleic acids. Consequently, this leads to an increased binding affinity for its target strands. Additionally, PNAs are extremely stable with respect to the various nucleases and proteases found in living organisms, which often makes the use of PNAs desirable for in vivo applications. Ordinary solid-phase peptide synthesis methods can be used for PNA oligomer synthesis methods. See, e.g., Nielsen et al., (1991) Science, 254:1497 for additional discussion of PNAs. When PNAs are employed in therapeutic applications, the mechanism by which the desired result is achieved is typically via translational arrest.

### II.B. Translocation Peptide

A composition of the present invention comprises a translocation peptide in addition to a modulation molecule. A function of a translocation peptide is to direct the composition, particularly the modulation molecule, to a particular site, at which, when the invention is employed in vivo, the association of the modulation molecule with one or more chemical entities disposed at the targeted site is known or suspected to provide a therapeutic benefit.

A translocation peptide can be of any length and composition, with the proviso that the translocation peptide is known or suspected to target a particular biological structure. A “biological structure” can be any structure that is normally disposed in an organism, and includes both desired and undesired structures. Examples of biological structures include various tissues, including tumor and adipose tissue (see, e.g., Kolonin et al., (2004) Nature Med. 10(6):625-632 and Rupnick et al., (2002) Proc. Natl. Acad. Sci. 99(16):10730-35), and proteins. In one embodiment of the invention, a translocation peptide is directed to the vasculature of adipose tissue.

A translocation peptide can direct a composition to a particular site using any one or more mechanisms. For example, a translocation peptide can have the ability to steer a composition to a molecule that is expressed on the surface of a cell. This can be achieved, in one embodiment, by the recognition of, and/or association with, a cell surface protein and the translocation peptide. Endocytosis of the cell surface protein will result in the internalization of the translocation peptide and its associated composition. Therefore, in this embodiment, the translocation peptide can both steer the composition to a particular site, such as a particular type of cell, and subsequently translocate the composition across the cell membrane, thereby fulfilling two roles: steering the composition to a desired location or type of cell and also facilitating the internalization of the composition by the cell. In another embodiment, the translocation peptide can facilitate the insertion and/or translocation of the composition across a cell membrane.

A translocation peptide can comprise any of the twenty standard amino acids, as well as modified amino acids, as described herein. Additionally, a translocation peptide can comprise standard peptide bonds, or any unique, non-standard chemistries. A translocation peptide can be generated using standard solid phase peptide synthesis techniques or it can be isolated from a biological source. In either case, a translocation peptide can be a cleavage product of a larger amino acid sequence.

### II.C. Optional Components

The core elements of a composition of the present invention include a translocation peptide and a modulation molecule, however a composition can further comprise additional optional components. Representative examples of additional components are a linker and a detectable label. The nature of additional components can depend on the application in which the composition is being employed. For example, in some cases it may be desirable to include various components designed to increase the effective in vivo lifetime of a composition.

### II.C.1. Linker

A composition of the present invention can optionally comprise a linker, which preferably spans and joins the modulation molecule and the translocation peptide. A linker can serve any one or more functions. For example, a linker can simply act as a spacer to separate the various components of a composition of the present invention, which can be beneficial in certain conditions in which the selection of a particular modulation molecule/translocation peptide pair requires consideration of steric factors. Alternatively, a linker can impart a degree of functional stability to the composition. A linker can serve in one or both of these capacities, or can be selected to address another consideration.

A linker can comprise any chemical entity. For example, a linker can be a peptide comprising any amino acid sequence, and can be of any length. Single amino acids can serve as linkers. Both standard and non-standard amino acids can be employed. PNAs can also be employed in a linker.

In another example, a linker can comprise modified or unmodified nucleotides, nucleosides; polymers; sugars and other carbohydrates; polyethers, e.g., polyethylene glycols, polyalcohols, polypropylenes, propylene glycols, mixtures of ethylene and propylene glycols; polyalkylamines; polyamines such as spermidine; polyesters such as poly(ethylacrylate); polyphosphodiesters; and alkylenes.

A linker can comprise one or more atoms linked by chemical bonds, such as one or more functional groups. When a linker is one or more atoms linked by chemical bonds, examples of linkers that can be employed in a composition of the present invention include disulfide linkages, urethane linkages, ester linkages and amide linkages.

A linker can also comprise one or more nucleic acids or a nucleic acid sequence. When a linker is a nucleic acid sequence, the nucleic acid can be an extension of a modulation molecule nucleic acid sequence or it can be a nucleic acid molecule that is distinct from, and unrelated to, a modulation molecule nucleic acid sequence, e.g. a poly-G, poly-A, poly-T, poly-C, poly-U or poly-I sequence.

When a linker is a peptide, the peptide can be an extension of a modulation molecule peptide sequence, when the modulation molecule is a peptide, or an extension of the translocation peptide sequence. Alternatively, a peptide linker can be distinct from, and unrelated to, a modulation molecule peptide sequence or a translocation peptide sequence, for example an amino acid sequence that is not part of an amino acid sequence from which a translocation peptide was derived..

### II.C.2. Detectable Label

In some situations, it may be desirable to track a composition, either in vivo or in an in vitro experiment. In this way, a measure of the effectiveness of a given translocation peptide can be determined and the exact location of the composition can be determined at any point in time.

Any detectable label can be employed in a composition of the present invention. Examples include radiolabels and fluorophores, including TAMRA, BODIPY, cyanin derivatives such as Cy3 or Cy5, fluorescein isothiocyanate, Texas red, rhodamine, dansyl, umbelliferone or an ALEXA dye. Fluorophores can be detected, and thus a composition comprising a flurophore label tracked, using fluorescence microscopy, for example. Radiolabel such as ³H, ¹²⁵I, ¹⁵S, ¹⁴C, or ³²P can also be employed in the present invention. Chemiluminescent compounds such as luciferin, and 2,3-dihydrophthalazinediones (e.g., luminal) can also be employed as detectable labels. In some situations, a protein or peptide can be employed as a detectable label, such as biotin, which can be detected by monitoring the association of the biotin with streptavidin.

The particular label or detectable group included in a composition of the present invention is not a necessary aspect of the invention, as the inclusion of which is optional, as long as it does not significantly interfere with the efficacy of the composition in a therapeutic application or the association of a composition with a selected target. As noted, any detectable label can be employed, and a detectable label can comprise any material having a detectable physical or chemical property. Thus, a detectable label is any composition detectable by spectroscopic, photochemical, biochemical, immunochemical, electrical, optical or chemical means.

A detectable label may be coupled directly or indirectly to any component of a composition, according to methods well known in the art. Thus, a detectable label can be associated with a translocation peptide, a modulation molecule or a linker, if a linker is included in the composition. The association of a detectable label and a component of a composition can be at any point in the component, for example on an unligated end or at a position intermediate between the defined ends of a component. As indicated above, a wide variety of labels may be used, with the choice of label depending on sensitivity required, ease of conjugation with the component, stability requirements, and available instrumentation.

Non-radioactive labels are often attached by indirect means. Generally, a ligand molecule is covalently bound to the molecule. The ligand then binds to another molecule, which is either inherently detectable or covalently bound to a signal system, such as a fluorescent compound, or a chemiluminescent compound.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorophore with the appropriate wavelength of light and detecting the resulting fluorescence. The resulting fluorescence can be detected visually, by means of photographic film, by the use of electronic detectors such as charge coupled devices (CCDs) or photomultipliers, for example. Simple colorimetric labels can be detected simply by observing the color associated with the label.

### II.E. Constructing a Composition of the Present Invention

A composition of the present invention can be formed by joining a modulation molecule directly or indirectly to a translocation peptide. A detectable label can also be joined to any component of the composition. A composition, therefore can comprise a modulation molecule joined directly to a translocation peptide. When no additional components make up the compositions, the translocation peptide can be joined to the modulation molecule at either the N or C terminal end of the translocation peptide. Similarly, when the modulation molecule is a siRNA oligo, the oligo can be joined to the translocation peptide at either the 3′ or 5′ end. Summarily, there is no restriction on the orientation or position of the modulation molecule relative to the translocation peptide.

A modulation molecule can be joined to a translocation peptide using any chemistry. The precise chemistry employed will be a function of the chemical nature of the components to be joined. Stated another way, the chemical reaction(s) used to join components of the present invention will depend on the nature of modulation molecule; if a modulation molecule is a peptide, the components can be joined, e.g., via a peptide bond.

Specific chemistries that can be employed to join the components of a composition of the present invention include, but are not limited to, amide, urethane, amine and ester bonds; all of these chemistries are well-known to those of skill in the art.

A composition of the present invention can also comprise any additional optional components, such as a linker or a detectable label. When a linker is employed in a composition, the linker can be disposed between the translocation peptide and the modulation molecule. When the modulation molecule is a siRNA oligo and a linker is employed, the linker can join the oligo to the translocation peptide via the 5′ or 3′ end of the oligo. Further, the peptide can be joined on the sense or antisense strand of the duplex.

### II.F. Pharmaceutically Acceptable Formulations

A composition of the present invention can be disposed in a pharmaceutically acceptable formulation. This can be desirable when the composition is employed in a therapeutic application. Any milieu that facilitates the introduction of a composition of the present invention into a cell can be employed in whole or in part as a pharmaceutically acceptable formulation. Exemplary components of a pharmaceutically acceptable formulation include, but are not limited to, solvents or dispersants, coatings, anti-infective agents, isotonic agents, agents that mediate absorption time or release of the inventive polynucleotides and double stranded polynucleotides. Other examples of components that can be included as a pharmaceutically acceptable formulation include neutral or cationic liposomes, and molecules that have the ability to destabilize endosomes and thereby aid in delivery of liposome contents into a cell, including a cell's nucleus such as chloroquine and 1,2-dioleoyl-sn-glycero-3-phosphatidylethanolamine. Further examples of components that can be included in a pharmaceutically acceptable formulation include poly-L-lysine, polyalkylcyanoacrylate nanoparticles, polyethyleneimines, and any suitable PAMAM dendrimers (polyamidoamine); PAMAM dendrimers can comprise any core such as, for example, an ethylenediamine core, and any desired surface functional groups such as, for example, cationic and anionic functional groups, amines, and ethanolanines.

A pharmaceutical formulations of the present invention can be mixed with a physiologically acceptable carrier medium. Examples of physiologically acceptable carrier media include, but are not limited to, water, buffered water, saline solutions (e.g., normal saline or balanced saline solutions such as Hank's or Earle's balanced salt solutions), 0.4% saline, 0.3% glycine, and hyaluronic acid.

Pharmaceutically acceptable formulations include formulations for human and veterinary use. Methods for preparing pharmaceutical compositions of the invention are within the skill in the art, for example as described in Remington: The Science and Practice of Pharmacy, 20th ed., Lippincott, Williams & Wilkins. (2003), the entire teaching of which is herein incorporated by reference.

### III. Method of Treating a Condition

In one embodiment of the invention, a composition of the present invention can be employed as a therapeutic to treat a condition. In this role, the composition can modulate the expression of a particular selected target. The modulation can also be of the activity of the selected target itself or it can be of an activity that is mediated by the selected target.

Modulation of the selected target can be via any mechanism. For example, a composition of the present invention can be employed to down-regulate, silence or up-regulate the expression of the selected target; this modulation can be achieved via any mechanism. In another embodiment of the invention, the composition can achieve modulation by blocking the binding site of an expressed selected target. This mechanism is characteristic of a method by which an aptamer can achieve a desired result.

Techniques for delivering a composition of the present invention to the vicinity of a selected target include administration of the composition to a subject using a gene gun, electroporation, nanoparticles, micro-encapsulation.

The composition can be administered via parenteral or enteral administration routes. Representative enteral administration routes include oral, rectal, or intranasal delivery. Representative parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of neovascularization, for example by a catheter or other placement device (e.g., a corneal pellet or a suppository, eye-dropper, or an implant comprising a porous, non-porous, or gelatinous material); and inhalation.

In one embodiment of the present invention, the composition is employed in the treatment of, for example, obesity. In this embodiment, therefore, the composition can comprise a translocation peptide that is directed to sites having high levels of angiogenic tissue, such as a adipose tissue. The composition can also comprise an siRNA oligo in the role of the modulation molecule. The siRNA oligo can comprise an oligo that is known or suspected to down-regulate or silence expression of VEGF and/or VEGFR, via the known siRNA mechanism. The composition can optionally comprise a linker and/or a detectable label. The detectable label can be useful for tracking the location of the composition as it localizes at the targeted site.

In one embodiment of the present invention, the composition is employed in the treatment of, for example, a tumor. Tumors are characterized by high levels of VEGF and VEGFR expression. In this embodiment, therefore, the composition can comprise a translocation peptide that is directed to sites having high levels of angiogenic tissue, such as a tumor site. The composition can also comprise an siRNA oligo in the role of the modulation molecule. The siRNA oligo can comprise an oligo that is known or suspected to down-regulate or silence expression of VEGF and/or VEGFR, via the known siRNA mechanism. The composition can optionally comprise a linker and/or a detectable label. The detectable label can be useful for tracking the location of the composition as it localizes at the targeted site.

The composition can be administered to a subject in need thereof using any of the techniques described herein. For example, injections or infusions of the composition can be given at or near the site of neovascularization.

In practicing the methods of the present invention, a composition of the present invention can be administered in a single dose or in multiple doses. Where the administration of the composition of the invention is by infusion, the infusion can be a single sustained dose or can be delivered by multiple infusions. When a composition of the present invention is employed in the treatment of a tumor, in accordance with the methods described herein, injection of composition directly into the tissue at or near the site of neovascularization is often preferred. Multiple injections at or near the site of neovascularization can form aspects of a therapeutic regimen.

One of ordinary skill in the art can readily determine an appropriate dosage regimen for administering a composition of the present invention to a given subject in accordance with the methods described herein. For example, a composition can be administered to the subject once, such as by a single injection or deposition at or near the neovascularization site. Alternatively, composition can be administered to a subject multiple times daily or weekly. In a representative dosage regimen, the composition is injected at or near the site of neovascularization once a week for a predetermined weeks, although indefinite therapeutic regimes form another aspect of the present invention.

### EXAMPLES

The following Examples have been included to illustrate various exemplary modes of the invention. Certain aspects of the following Examples are described in terms of techniques and procedures found or contemplated by the inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

### Treatment of a Condition

A composition is prepared that can be employed in the treatment of a condition in which it is desired to down-regulate or silence the expression or activity of a gene implicated in the condition. In one example, the condition is the presence or growth of a tumor. In another example, the condition is obesity.

A composition comprising a vasculature translocation peptide comprising the amino acid sequence CKGGRAKDC (SEQ ID NO:1), and a nucleic acid targeted agent comprising an anti-VEGFR or anti-VEGF siRNA oligo, is prepared using appropriate chemical reactions. The composition can optionally comprise a linker and a detectable label.

The composition is administered to a subject in the vicinity of a site known or suspected to be undergoing vascularization, for example by injection, or by employing an osmotic minipump.

The effect of the composition on obesity, or tumor growth and/or angiogenesis, in the site of administration can be assayed using any means known in the art. In the case of obesity, a reduction in adipose tissue can be monitored in either whole body assays or by evaluating the quality and quantity of adipose tissue at a particular location. If the composition comprises a detectable label, the localization of the composition can be tracked using a technique appropriate to the detectable label.

### References

The references cited in the specification are incorporated herein by reference to the extent that they supplement, explain, provide a background for or teach methodology, techniques and/or compositions employed herein. All cited patents, including patent applications, and publications referred to in this application are herein expressly incorporated by reference. Also expressly incorporated herein by reference are the contents of all citations of GenBank accession numbers, LocusID, and other computer database listings, as well as the contents of the Sequence Listing associated herewith.

It will be understood that various details of the invention may be changed without departing from the scope of the invention. Furthermore, the foregoing description is for the purpose of illustration only.

What is claimed is:

1. A composition comprising a translocation peptide and a modulation molecule directed to a selected target.

2. The composition of claim 1, wherein the selected target is an angiogenic protein.

3. The composition of claim 2, wherein the angiogenic protein is selected from the group consisting of VEGF, a VEGFR, an integrin and an angiopoeitin.

4. The composition of claim 1, wherein the translocation peptide is a ligand for an endothelial cell-selective receptor.

5. The composition of claim 4, wherein the translocation peptide comprises CKGGRAKDC (SEQ ID NO:1).

6. The composition of claim 1, wherein the modulation molecule is selected from the group consisting of a double-stranded siRNA oligo, a PNA, a single-stranded antisense oligo, a peptide, an aptamer and a pyrrole-imidazole oligomer.

7. The composition of claim 1, wherein the selected target is one of VEGF, the VEGF receptor, basic fibroblast growth factor and integrin and an angiopoetin.

8. The composition of claim 1, further comprising a linker.

9. The composition of claim 9, wherein the linker is selected from the group consisting of a chemical linkage, a peptide linker, a nucleic acid linker and a PNA linker.

10. The composition of claim 10, wherein the chemical linkage is selected from the group consisting of a disulfide linkage, a urethane linkage, an ester linkage, and an amide linkage.

11. The composition of claim 1, further comprising a detectable reagent.

12. The composition of claim 12, wherein the detectable reagent is selected from the group consisting of a fluorophore, a radiolabel, a colorimetric reagent and a protein.

13. The composition of claim 13, wherein the fluorophore is selected from the group consisting of TAMRA, BODIPY, cyanin derivatives such as Cy3 or Cy5, fluorescein isothiocyanate, Texas red, rhodamine, dansyl, umbelliferone and an ALEXA dye.

14. A method of modulating one of (a) the activity of a selected target and (b) an activity mediated by a selected target, the method comprising administering to a subject in need thereof the composition of claim 1.

15. The method of claim 15, wherein the selected target is an angiogenic protein.

16. The method of claim 16, wherein the angiogenic protein is selected from the group consisting of VEGF, a VEGFR, an integrin and an angiopoeitin.

17. The method of claim 15, wherein the translocation peptide is a ligand for an endothelial cell-selective receptor.

18. The method of claim 18, wherein the translocation peptide comprises CKGGRAKDC (SEQ ID NO: 1).

19. The method of claim 15, wherein the modulation molecule is selected from the group consisting of a double-stranded siRNA oligo, a PNA, a single-stranded antisense oligo, a peptide, an aptamer and a pyrrole-imidazole oligomer.

20. The method of claim 15, wherein the selected target is one of VEGF, the VEGF receptor, basic fibroblast growth factor, an integrin and an angiopoeitin.

21. The method of claim 15, wherein the composition further comprises a linker.

22. The method of claim 23, wherein the linker is selected from the group consisting of a chemical linkage, a peptide linker, a nucleic acid linker and a PNA linker.

23. The method of claim 24, wherein the chemical linkage is selected from the group consisting of a disulfide linkage, a urethane linkage, an ester linkage, and an amide linkage.

24. The method of claim 15, wherein the composition further comprises a detectable reagent.

25. The method of claim 26, wherein the detectable reagent is selected from the group consisting of a fluorophore, a radiolabel, a calorimetric reagent and a protein.

26. The method of claim 27, wherein the fluorophore is selected from the group consisting of TAMRA, BODIPY, cyanin derivatives such as Cy3 or Cy5, fluorescein isothiocyanate, Texas red, rhodamine, dansyl, umbelliferone and an ALEXA dye.

27. A method of treating a condition comprising administering to a subject in need thereof the composition of claim 1.

28. The method of claim 29, wherein the selected target is an angiogenic protein.

29. The method of claim 30, wherein the angiogenic protein is selected from the group consisting of VEGF, a VEGFR, an integrin and an angiopoeitin.

30. The method of claim 29, wherein the translocation peptide is a ligand for an endothelial cell-selective receptor.

31. The method of claim 32, wherein the translocation peptide comprises CKGGRAKDC (SEQ ID NO:1).

32. The method of claim 29, wherein the modulation molecule is selected from the group consisting of a double-stranded siRNA oligo, a PNA, a single-stranded antisense oligo, a peptide, an aptamer and a pyrrole-imidazole oligomer.

33. The method of claim 29, wherein the selected target is one of VEGF, the VEGF receptor, basic firbroblast growth factor, an integrin and an angiopoeitin.

34. The method of claim 29, wherein the composition further comprises a linker.

35. The method of claim 37, wherein the linker is selected from the group consisting of a chemical linkage, a peptide linker, a nucleic acid linker and a PNA linker.

36. The method of claim 38, wherein the chemical linkage is selected from the group consisting of a disulfide linkage, a urethane linkage, an ester linkage, and an amide linkage.

37. The method of claim 29, wherein the composition further comprises a detectable reagent.

38. The method of claim 40, wherein the detectable reagent is selected from the group consisting of a fluorophore, a radiolabel, a colorimetric reagent and a protein.

39. The method of claim 41, wherein the fluorophore is selected from the group consisting of TAMRA, BODIPY, cyanin derivatives such as Cy3 or Cy5, fluorescein isothiocyanate, Texas red, rhodamine, dansyl, umbelliferone and an ALEXA dye.

40. A composition comprising a vasculature translocation peptide comprising the amino acid sequence of CKGGRAKDC (SEQ ID NO:1) and an siRNA oligo directed against one of VEGF and a VEGF receptor.
